(19) United States
(12) Patent Application Publication
(10) Pub. No.: US 2026/0221285 A1
(43) Pub. Date: Jul. 30, 2026

(54) SYSTEMS AND METHODS FOR BIOLOGICAL AGE PREDICTION

(71) Applicant: LOMA LINDA UNIVERSITY, Loma Linda, CA (US)

(72) Inventors: Trevor LOHMAN, Loma Linda, CA (US); Liang JI, Loma Linda, CA (US)

(73) Assignee: LOMA LINDA UNIVERSITY, Loma Linda, CA (US)

(21) Appl. No.: 19/147,500

(22) PCT Filed: Jan. 12, 2024
(86) PCT No.: PCT/US2024/011448

§ 371 Date: Jul. 11, 2025

Related U.S. Application Data
(60) Provisional application No. 63/479,693, filed on Jan. 12, 2023.

(51) Int. Cl. G16H 50/30; G16H 10/40; G16H 10/60

(52) U.S. Cl. CPC G16H 50/30; G16H 10/40; G16H 10/60

(57) ABSTRACT

Provided here are methods and systems to generate a biological age based on one or more variables. In an embodiment, a method for generating a biological age may include receiving one or more of hematological variables or demographic variables corresponding to a user. The method may include applying each one of the one or more of hematological variables or demographic variables to a model to thereby generate a score. The method may include determining a biological age for the user based on score.

### TECHNICAL FIELD

The disclosure relates to methods and systems for generating a biological age for a user based on one or more variables. More specifically, the methods and systems may apply one or more of demographic variables, biometric variables, or hematological variables obtained or received from one or more sources to a model to generate a score, and, based on the score and, if available, other previously determined biological ages, generate a biological age.

### BACKGROUND

Implementing new biomarkers that may indicate age and interpreting the data that those biomarkers generate poses many challenges. There are now numerous categories of biological age predictors, such as DNA methylation based models, composite models of hematological biomarkers, transcriptomic predictors of biological age, and functional measures of biological age, and among others.

As models that predict biological age become more informative and more accurate, those become more complex and more nuanced. Such issues are a likely consequence of the complex, multi-dimensional biological phenomenon that the models are designed to measure. While this complexity may aid in elucidation of the mechanisms of aging, the clinical value of such models remains uncertain. To the typical healthcare provider, the models and the data generated therefrom may not appear useful in guiding clinical practice and the complexity of the data interpretation makes them unsuited as patient education tools.

### SUMMARY

Provided here are systems and methods to address these shortcomings of the art and provide other additional or alternative advantages. Provided here are methods and systems for generating a biological age for a user based on one or more variables. More specifically, the methods and systems may apply one or more of demographic variables, biometric variables, or hematological variables obtained or received from one or more sources to a model to generate a score, and, based on the score and, if available, other previously determined biological ages, generate a biological age.

As noted, there exists a need for a biomarker of aging that generates easy to interpret data. Particularly, from biomarkers which are routinely or typically available in the healthcare setting instead of specialized data such as CpG site methylation, telomere length, or mRNA abundance, or other specialized variables. Such specialized data may be difficult to obtain.

As such, new biomarkers of aging are utilized and are intended to serve as an intuitive representation of clinical blood marker/lifestyle factor derived risk. The biomarkers are not biomarkers of aging per se, as the biomarkers do not predict chronological age or the discrepancy between predicted and actual age (for example, age-acceleration). Instead, the additive model described herein estimates relative risk in a manner that is easily interpreted and patient-relevant, potentially serving as a useful patient education tool and providing an estimation of a predicted or biological age and/or of a predicted or biological age versus actual age. The estimated relative risk may provide further context to laboratory values often present in a medical chart.

The additive risk model described herein may utilize biomarkers found in routinely ordered blood panels (for example, metabolic panel, lipid panel, and/or differential CBC, among other blood panels) to report relative mortality risk in terms of a lifespan acceleration value (for example, the difference between a predicted or biological age versus actual age) measured in years. The result is a metric derived from biomarkers, which may otherwise be meaningless to a patient, that is a personally relevant biological age metric.

In an embodiment, a user may receive such a biological age after inputting one or more of hematological variables, demographic variables, or biometric variables into a device. Such a device may be a computing device, a mobile device, a wearable mobile device, and/or a device positioned at a hospital or medical facility, among other devices. The device may receive such inputs directly or indirectly. For example, the device may be included in or with, integrated with, and/or comprise a hematological analyzer. Further, the device may include one or more sensors and/or a user interface. Further still, the device may include a communications circuitry. The communications circuitry may connect to various data sources to receive data or variables (for example, reception of such data from one or more databases, electronic health records, and/or a hematological analyzer, among other devices). Thus, the device may directly or indirectly receive the variables (for example, hematological variables, demographic variables, and/or biometric variables).

To generate such a biological age, a user may provide or be tested to produce hematological variables and/or a blood panel. For example, the user may provide a blood sample for analysis via a hematological analyzer. The hematological analyzer may produce a number of different hematological variables. The device may receive the hematological variables directly or indirectly from the hematological analyzer. In other words, the device may be included with the hematological analyzer (for example, the hematological variables received as the hematological variables become available), may be in signal communication with the hematological analyzer (for example, the hematological variables transmitted over the connection from the hematological analyzer to the device), or may include a user interface configured to receive the input (for example, a user may enter the hematological variables into the user interface). The hematological variables may include a glycohemoglobin percentage of a user's blood sample, a triglyceride content of the user's blood sample, a total cholesterol of the user's blood sample, a uric acid content of the user's blood sample, a red cell distribution width percentage of the user's blood sample, a white blood cell count of the user's blood sample, a mean cell volume of the user's blood sample, a mean platelet volume quintile 1 of the user's blood sample, a mean platelet volume quintile 2 of the user's blood sample, a mean platelet volume quintile 3 of the user's blood sample, a mean platelet volume quintile 4 of the user's blood sample, a mean platelet volume quintile 5 of the user's blood sample, a lactate dehydrogenase content of the user's blood sample, an alkaline phosphatase content of the user's blood sample, a potassium content of the user's blood sample, a gamma-glutamyl transferase of the user's blood sample, a blood urea nitrogen creatine ratio of the user's blood sample, an alanine transaminase content of the user's blood sample, an aspartate aminotransferase content of the user's blood sample, a chloride quintile 1 of the user's blood sample, a chloride quintile 2 of the user's blood sample, a chloride quintile 3 of the user's blood sample, a chloride quintile 4 of the user's blood sample, or a chloride quintile 5 of the user's blood sample.

Next, the user may input demographic variables into the device. Such demographic variables may be entered in as part of an initiation process or to update existing variables. The demographic variables may be entered directly into the device. The demographic variables may be transmitted or requested from one or more different databases (for example, a database storing user or patient data and/or electronic health records, among other databases). The demographic variables may include whether a user smoked a selected number of cigarettes in the user's lifetime, a gender of the user, a body mass index of the user, a current age of the user, a weight of the user, or a height of the user.

The device may also utilize biometric variables. For example, the device may include sensors or the device may receive such biometric variables via a connection to a sensor or device configured to measure or obtain such biometric variables. The sensors or other devices may measure blood pressure, heart rate, and/or other variables. In another embodiment, the sensors or other devices may measure or obtain such variables continuously or substantially continuously. The sensors or other devices may be or may be included in a wearable device. Such variables may include activity, exercise, biometric variables during such activities or exercise, or other biometric variables. Other variables may be utilized, such as current prescription medications, current exercise, current diet, or drug use, among others.

Once the device has received one or more of the hematological variables, the demographic variables, and/or the biometric variables, the device may determine whether previous variables exist. In a non-limiting example, the device may receive hematological variables and utilize previously entered demographic variables and/or biometric variables, in addition to the received hematological variables, to generate the biological age. Once a preselected amount of variables are available, the device may apply the variables to a model or trained model. Such application of the variables to the model or trained model may produce a score. In an embodiment, a score may be produced for each variable. The score or scores may each be utilized by the device to determine a biological age. In such embodiments, each score may be utilized to determine a particular number, that number representing an amount of years or a portion of a year. The aggregate of those numbers may represent the total age acceleration. When added to the user's current age, the resulting number may be the biological age. Thus, the use of such a device and model may produce an easily understood context for each variable and how such results increase age acceleration. Further, such a device can be used in many different scenarios, and may offer continuous updates over time. Further still, the device may be utilized by a doctor or medical professional to determine treatment regimens to decrease such age acceleration based on corresponding variables.

Finally, the resulting biological age, in addition to or rather than the gathered variables, may be utilized to determine or generate a treatment regimen. The treatment regimen may include prescription of a specified medicine, determination of a particular diet and/or exercise, and/or other treatment.

Accordingly, an embodiment of the disclosure is directed to a method for generating a biological age. The method may include receiving one or more of hematological variables or demographic variables corresponding to a user. The method may include applying each one of the one or more of hematological variables or demographic variables to a model to thereby generate a score. The method may include determining a biological age for the user based on score.

In an embodiment, the model may comprise one or more of a composite model, a cox proportional hazards regression model, an unsupervised model, a supervised model, or a machine learning model.

In an embodiment, the method may include, prior to application of each of the one of the one or more of hematological variables or demographic variables to the model, pre-processing each one of the one or more of hematological variables or demographic variables to a model. Pre-processing may include formatting the one or more of hematological variables or demographic variables to a format applicable to the model and filtering the one or more of hematological variables or demographic variables to remove non-applicable or outlying variables or anomalies.

In another embodiment, generation of the score may include generation of an age ratio metric for each variable, each age ratio metric defined by a beta coefficient of each variable divided by a beta coefficient of age. Determination of the biological age may be based on each age ratio metric and a current age of the user. Each age ratio metric may be based on variables comprising one or more of whether a user smoked a selected number of cigarettes in the user's lifetime, a gender of the user, a body mass index of the user, a current age of the user, a glycohemoglobin percentage of a user's blood sample, a triglyceride content of the user's blood sample, a total cholesterol of the user's blood sample, a uric acid content of the user's blood sample, a red cell distribution width percentage of the user's blood sample, a white blood cell count of the user's blood sample, a mean cell volume of the user's blood sample, a mean platelet volume quintile 1 of the user's blood sample, a mean platelet volume quintile 2 of the user's blood sample, a mean platelet volume quintile 3 of the user's blood sample, a mean platelet volume quintile 4 of the user's blood sample, a mean platelet volume quintile 5 of the user's blood sample, a lactate dehydrogenase content of the user's blood sample, an alkaline phosphatase content of the user's blood sample, a potassium content of the user's blood sample, a gamma-glutamyl transferase of the user's blood sample, a blood urea nitrogen creatine ratio of the user's blood sample, an alanine transaminase content of the user's blood sample, an aspartate aminotransferase content of the user's blood sample, a chloride quintile 1 of the user's blood sample, a chloride quintile 2 of the user's blood sample, a chloride quintile 3 of the user's blood sample, a chloride quintile 4 of the user's blood sample, or a chloride quintile 5 of the user's blood sample. The method of claim 1, wherein the model is a trained model. The training data for training the model may include one or more publicly available datasets, each of the one or more publicly available datasets including hematological variables and demographic variables. Each of the one or more publicly available datasets include cause of death data for some subjects. Further the method may include validating the trained model via a cross validation procedure with data from the one or more publicly available datasets which include hematological variables, demographic variables, and cause of death data.

In an embodiment, the biological age may be based on one or more previously received hematological data, previously received demographic data, or a previously determined biological age. The method may include determining a treatment regimen based on the biological age and a user's current age.

Another embodiment of the disclosure is directed to a method for generating a biological age. The method may include receiving one or more of one or more hematological variables or one or more demographic variables corresponding to a user. The method may include determining availability of one or more of (1) previously received hematological variables, (2) previously received demographic variables corresponding to the user, or (3) previously generated biological ages. The method may include applying each one of the one or more of (1) one or more hematological variables, (2) one or more demographic variables, (3) previously received hematological variables, or (4) previously received demographic variables corresponding to the user to thereby generate a new biological age of the user. The method may include determining an updated biological age based on the new biological age and previously generated biological ages.

In an embodiment, a hematological analyzer may generate the one or more hematological variables based on a blood sample of the user. In another embodiment, the user may provide the one or more demographic variables via a user interface. The method may include, in response to determination of the updated biological age: determining a treatment regimen based on the updated biological age, the new biological age, and the previously generated biological age; and displaying one or more of the updated biological age, the new biological age, or the previously generated biological age. The method may include displaying the one or more hematological variables or one or more demographic variables. The treatment regimen may be based on the one or more hematological variables or one or more demographic variables. The method may include displaying the difference between the biological age and a current age of the user.

Another embodiment of the disclosure is directed to an apparatus for generating a biological age, the apparatus comprising: at least one biometric sensor configured to substantially continuously monitor and gather biometric data of a user; a user interface; a processor; and a non-transitory machine-readable storage medium storing processor-executable instructions that, when executed by the processor, cause the at least one processor to: in response to a determination that a user profile includes an existing biological age: apply gathered biometric data to a model to thereby generate a new biological age, determine an updated biological age based on the new biological age and an existing biological age, and display the updated biological age to the user interface.

In an embodiment, the apparatus may comprise a mobile device. The mobile device comprises a wearable device.

In an embodiment, the non-transitory machine readable storage medium may include executable instructions, when executed by the processor, to cause the processor to: request demographic variables of the user; in response to reception of the demographic variables, apply received demographic variables, in addition to the gathered biometric data, to the model to thereby generate the new biological age; and determine the updated biological age based on the new biological age and the existing biological age. The non-transitory machine readable storage medium may include executable instructions, when executed by the processor, to cause the processor to, prior to determination of the updated biological age and if the existing biological age is not available: prompt the user to submit a current actual age; and in response to the reception of the current actual age, determine the updated biological age based on the new biological age and the current actual age. The non-transitory machine readable storage medium may include executable instructions, when executed by the processor, to cause the processor to, prior to determination of the updated biological age: prompt the user to initialize the apparatus, wherein initialization includes one or more of entry of demographic variables or establishment of a connection with a data source including hematological variables. The non-transitory machine readable storage medium may include executable instructions, when executed by the processor, to cause the processor to, prior to determination of the updated biological age, apply one or more of the demographic variables or hematological variables, in addition to gathered biometric data, to the model to determine the new biological age.

Another embodiment of the disclosure is directed to an apparatus for generating a biological age. The apparatus may include a user interface configured to receive demographic variables corresponding to each of a plurality of patients. The apparatus may include a communications circuitry configured to connect with an analyzer. The analyzer may be configured to receive hematological variables corresponding to each of the plurality of patients. The apparatus may include a processor. The apparatus may include a non-transitory machine-readable storage medium storing processor-executable instructions that, when executed by the processor, cause the at least one processor to: apply received demographic variables and received hematological variables for one of the plurality of patients to a model to thereby generate a score; and determine a biological age based on the score.

In an embodiment, the apparatus may be positioned proximate a medical practitioners office or a hospital. In another embodiment, the user interface may be configured to display the biological age. In an embodiment, the non-transitory machine readable storage medium may include executable instructions, when executed by the processor, to cause the processor to determine a treatment regimen based on the biological age, and wherein the user interface is configured to display the treatment regimen.

Another embodiment of the disclosure is directed to an apparatus for generating a biological age. The apparatus may include a user interface configured to receive demographic variables corresponding to each of a plurality of patients. The apparatus may include a hematological analyzer configured to receive a biological sample and generate hematological variables. The apparatus may include a processor. The apparatus may include a non-transitory machine-readable storage medium storing processor-executable instructions that, when executed by the processor, cause the at least one processor to: apply received demographic variables and hematological variables for one of the plurality of patients to a model to thereby generate a score; and determine a biological age based on the score.

In an embodiment, the user interface may be configured to receive biometric measurements. The biometric measurements may include one or more of weight, height, or blood pressure. The generation of the score may further be based on application of the biometric measurements to the model.

In another embodiment, the user interface may receive or may be configured to receive the demographic variables from electronic health records. In another embodiment, a database may be in signal communication with the user interface and may store the electronic health records.

Another embodiment of the disclosure is directed to a system for generating a biological age. The system may include a hematological analyzer configured to receive a biological sample and generate hematological variables. The system may include a device in signal communication with the hematological analyzer. The device may include a user interface configured to receive demographic variables corresponding to each of a plurality of patients. The device may include a processor. The device may include a non-transitory machine-readable storage medium storing processor-executable instructions that, when executed by the processor, cause the at least one processor to: apply hematological variables from the hematological analyzer and received demographic variables for one of the plurality of patients to a model to thereby generate a score, and determine a biological age based on the score.

In another embodiment, a score is generated for each hematological variable and each demographic variable. The non-transitory machine readable storage medium may include executable instructions, when executed by the processor, to cause the processor to aggregate each score to form an aggregate score. The biological may be determined based on the aggregate score.

Still other aspects and advantages of these embodiments and other embodiments, are discussed in detail herein. Moreover, it is to be understood that both the foregoing information and the following detailed description provide merely illustrative examples of various aspects and embodiments, and are intended to provide an overview or framework for understanding the nature and character of the claimed aspects and embodiments. Accordingly, these and other objects, along with advantages and features herein disclosed, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

These and other features, aspects, and advantages of the disclosure will become better understood with regard to the following descriptions, claims, and accompanying drawings. It is to be noted, however, that the drawings illustrate only several embodiments of the disclosure and, therefore, are not to be considered limiting of the disclosure's scope.

FIG. 1 is a block diagram of a system to train a machine learning model to determine a biological age, according to an embodiment of the present disclosure.

FIG. 2 is a flowchart of a method to select training data to train the machine learning model for determining a biological age, according to an embodiment of the present disclosure.

FIG. 3A and FIG. 3B are block diagrams of an apparatus with a machine learning model to determine a biological age, according to an embodiment of the present disclosure.

FIG. 4 is a block diagram of a system to determine a biological age, according to an embodiment of the present disclosure.

FIG. 5 is another block diagram of a system to determine a biological age, according to an embodiment of the present disclosure.

FIG. 6 is a flowchart of a method to utilize the machine learning model for determining a biological age, according to an embodiment of the present disclosure.

FIG. 7A and FIG. 7B illustrate a receiver operating characteristic (ROC) curve and an area under the ROC (AUC ROC) curve regarding determination of a biological age.

FIG. 7C illustrates an AUC ROC curve regarding vital status follow-up.

### DETAILED DESCRIPTION

So that the manner in which the features and advantages of the embodiments of the systems and methods disclosed herein, as well as others, which will become apparent, may be understood in more detail, a more particular description of embodiments of systems and methods briefly summarized above may be had by reference to the following detailed description of embodiments thereof, in which one or more are further illustrated in the appended drawings, which form a part of this specification. It is to be noted, however, that the drawings illustrate only various embodiments of the embodiments of the systems and methods disclosed herein and are therefore not to be considered limiting of the scope of the systems and methods disclosed herein as it may include other effective embodiments as well.

Implementing new biomarkers that may indicate age and interpreting the data that those biomarkers generate poses many challenges, as noted above. Additionally, biomarkers typically used to determine a biological age are complicated to obtain and/or analyze.

Thus, provided herein are methods and systems for generating a biological age or biological age prediction for a user based on one or more variables. More specifically, the methods and systems may apply one or more of demographic variables, biometric variables, or hematological variables obtained or received from one or more sources to a model to generate a score, and, based on the score and, if available, other previously determined biological ages, generate a biological age or biological age prediction.

As noted, there exists a need for a biomarker of aging that generates easy to interpret data. Particularly, from biomarkers which are routinely or typically available in the healthcare setting instead of specialized data such as CpG site methylation, telomere length, or mRNA abundance, among other specialized variables. Such specialized data may be difficult to obtain.

As such, new biomarkers of aging are utilized and are intended to serve as an intuitive representation of clinical blood marker/lifestyle factor derived risk. The biomarkers are not biomarkers of aging per se, as the biomarkers do not predict chronological age or the discrepancy between predicted and actual age (for example, age-acceleration). Instead, the additive model described herein estimates relative risk in a manner that is easily interpreted and patient-relevant, potentially serving as a useful patient education tool and providing an estimation of a biological age or predicted biological age and/or of a biological age or predicted biological age versus actual age. The estimated relative risk may provide further context to laboratory values often present in a medical chart.

The additive risk model described herein may utilize biomarkers found in routinely ordered blood panels (for example, metabolic panel, lipid panel, and/or differential CBC, among other blood panels), as well as utilizing demographic variables (for example, height, weight, body mass index (BMI), and/or cigarettes smoked, among other demographic variables) and/or biometric variables (for example, amount of activity, steps taken per day, heart rate or pulse, and/or blood pressure, among other biometric variables), to report relative mortality risk in terms of a lifespan acceleration value (for example, the difference between a predicted or biological age versus actual age) measured in years. The result is a metric derived from biomarkers and/or other variables or data, which may otherwise be meaningless to a patient, that is a personally relevant biological age metric.

In an embodiment, a user may receive such a biological age after inputting one or more of hematological variables, demographic variables, or biometric variables into a device. Such a device may be a computing device, a mobile device, a wearable mobile device, and/or a device positioned at a hospital or medical facility, among other devices. The device may receive such inputs directly or indirectly. For example, the device may be included in or with, integrated with, and/or comprise a hematological analyzer. Further, the device may include one or more sensors and/or a user interface. Further still, the device may include a communications circuitry. The communications circuitry may connect to various data sources to receive data or variables (for example, databases, electronic health records, and/or a hematological analyzer). Thus, the device may directly or indirectly receive the variables (for example, hematological variables, demographic variables, and/or biometric variables).

To generate such a biological age or biological age prediction, a user may provide or be tested to produce hematological variables. For example, the user may provide a blood sample for analysis via a hematological analyzer. The hematological analyzer may produce a number of different hematological variables. The device may receive the hematological variables directly or indirectly from the hematological analyzer. In other words, the device may be included with the hematological analyzer (for example, the hematological variables received as the hematological variables become available), may be in signal communication with the hematological analyzer (for example, the hematological variables transmitted over the connection from the hematological analyzer to the device), or may include a user interface configured to receive the input (for example, a user may enter the hematological variables into the user interface). The hematological variables may include a glycohemoglobin percentage of a user's blood sample, a triglyceride content of the user's blood sample, a total cholesterol of the user's blood sample, a uric acid content of the user's blood sample, a red cell distribution width percentage of the user's blood sample, a white blood cell count of the user's blood sample, a mean cell volume of the user's blood sample, a mean platelet volume quintile 1 of the user's blood sample, a mean platelet volume quintile 2 of the user's blood sample, a mean platelet volume quintile 3 of the user's blood sample, a mean platelet volume quintile 4 of the user's blood sample, a mean platelet volume quintile 5 of the user's blood sample, a lactate dehydrogenase content of the user's blood sample, an alkaline phosphatase content of the user's blood sample, a potassium content of the user's blood sample, a gamma-glutamyl transferase of the user's blood sample, a blood urea nitrogen creatine ratio of the user's blood sample, an alanine transaminase content of the user's blood sample, an aspartate aminotransferase content of the user's blood sample, a chloride quintile 1 of the user's blood sample, a chloride quintile 2 of the user's blood sample, a chloride quintile 3 of the user's blood sample, a chloride quintile 4 of the user's blood sample, or a chloride quintile 5 of the user's blood sample.

Next, the user may input demographic variables into the device. Such demographic variables may be entered in as part of an initiation process or to update existing variables. The demographic variables may be entered directly into the device. The demographic variables may be transmitted or requested from one or more different databases (for example, a database storing user or patient data, and/or electronic health records). The demographic variables may include whether a user smoked a selected number of cigarettes in the user's lifetime, a gender of the user, a body mass index of the user, a current age of the user, a weight of the user, or a height of the user. In another embodiment, a large language model and/or other types of machine learning models may be utilized to process the demographic variables to generate the biological age or an output to be included in generating the biological age.

The device may also utilize biometric variables. For example, the device may include sensors or the device may receive such biometric variables via a connection to a sensor or another device configured to measure or obtain such biometric variables. The sensors or other devices may measure blood pressure, heart rate or pulse, and/or other variables. In another embodiment, the sensors or other devices may measure or obtain such variables continuously or substantially continuously. The sensors or other devices may be or may be included in a wearable device. Such variables may include activity, exercise, biometric variables during such activities or exercise, or other biometric variables. Other variables may be utilized, such as current prescription medications, current exercise, current diet, or drug use, among others.

Once the device has received one or more of the hematological variables, the demographic variables, and/or the biometric variables, the device may determine whether previous variables exist. In a non-limiting example, the device may receive hematological variables and utilize previously entered demographic variables and/or biometric variables, in addition to the received hematological variables, to generate the biological age or biological age prediction. Once a preselected amount of variables are available, the device may apply the variables to a model or trained model. Such application of the variables to the model or trained model may produce a score. In an embodiment, a score may be produced for each variable. The score or scores may each be utilized by the device to determine a biological age or biological age prediction. In such embodiments, each score may be utilized to determine a particular number, that number representing an amount of years or a portion of a year. The aggregate of those numbers may represent the total age acceleration. When added to the user's current age, the resulting number may be the biological age or biological age prediction. Thus, the use of such a device and model may produce an easily understood context for each variable and how such results increase age acceleration. Further, such a device can be used in many different scenarios, and may offer continuous updates over time. Further still, the device may be utilized by a doctor or medical professional to determine treatment regimens to decrease such age acceleration based on corresponding variables.

As noted, the device may include or be connected to a model. The model (for example, an additive risk model) may be trained to produce a score for one or more variables. Each score may indicate an amount of years or a portion of a year that a particular variable increases an actual age. Thus, the training may produce a model which, when variables are applied thereto, produces scores each score indicating an accelerated age amount (for example, the amount a variable increases an actual age). The model may be trained with data including known outcomes. The data may include variables and mortality data (for example, such as the age of the patient at the time of death) corresponding to a patient. One or more data sources may be utilized for such training. For example, National Health and Nutrition Examination Survey (NHANES) data may be utilized. Other data may include other publicly related health records or data and/or privately gathered, non-public data. Further, the model may be continuously trained or retrained based on mortality of users and/or patients and corresponding generated biological ages.

Finally, the resulting biological age or biological age prediction, in addition to or rather than the gathered variables, may be utilized to determine or generate a treatment regimen. The treatment regimen may include prescription of a specified medicine, determination of a particular diet and/or exercise, and/or other treatment.

### EXAMPLES

### Example 1

### Systems and Methods

The embodiments or examples disclosed herein may include training and utilization of a model to determine a biological age or biological age prediction and treatment regimen based on one or more of hematological variables, demographic variables, or biometric variables.

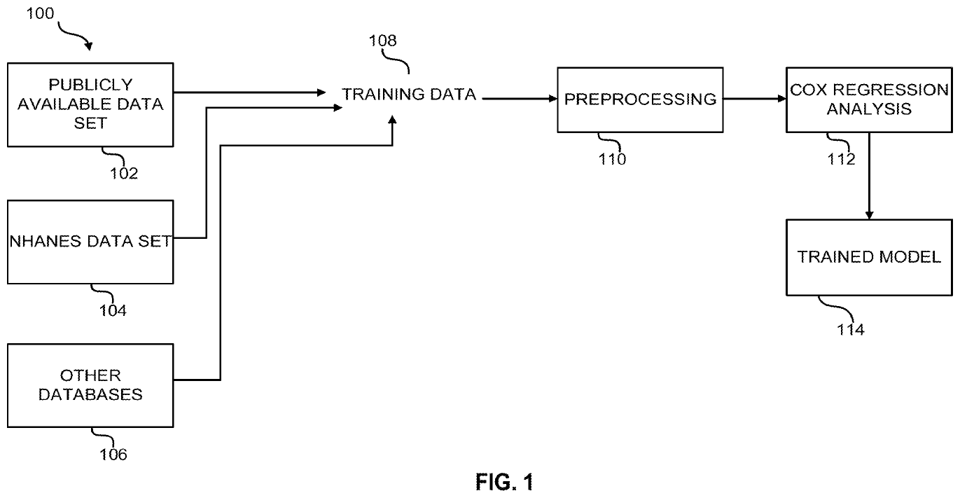

A system to train or generate the model, trained model 114, or classifier (for example, a statistical model, probabilistic model, trained machine learning model, and/or other classifier to accept an input and produce an output), is illustrated in FIG. 1. The system 100 may accept or receive data from various databases or sources as training data 108. Databases or other sources providing training data 108 may include a publicly available data set 102, a NHANES data set 104, and/or other databases 106 (for example, a hospital database, and/or a medical facility database) including relevant variables and/or mortality data. The data may be received or provided directly from the databases or via a client or user interface.

The training data 108 may include a number of user or patient variables, such as demographic variables, hematological variables, biometric variables, and/or mortality data. Each user's or patient's mortality data may be indicated by the age at which the corresponding user or patient has died. The remaining variables may be indicated by numbers, units, and/or labels indicating the corresponding variable. Further, the training data may include data for a number of subjects, for example, 100 subjects, 500 subjects, 1000 subjects, 10,000 subjects, and more. In another embodiment, other variables may include survey data, such as surveys covering reported physical activity; questionaries covering cognitive status, depression, and/or anxiety; and/or other survey data from publicly accessible databases or datasets such as databases or datasets located at the National Health and Nutrition Examination Survey at the Center for Disease Control. In yet another embodiment, the biometric variables may include, but are not limited to, previously recorded or logged accelerometer data, vital sign data, carcinogen concentration data, and/or body composition data.

After reception of the training data 108 at the system 100, the training data 108 may be transmitted to a preprocessing engine, circuitry, or module (for example, see preprocessing 110). Preprocessing 110 may include removing data including non-public records (for example, indicated by a label, flag, bit, or other indicator) and/or removing sets of variables for corresponding users that are missing one or more different variables. Preprocessing 110 may include reformatting variables for each user or patient, normalizing the variables for each user or patient, and/or weighting selected variables for each set of variables for each user or patient. In another embodiment, preprocessing 110 may include determining variables based on received data (for example, the training data 108 and/or other data). For example, blood urea nitrogen (BUN)/Creatinine data may be utilized to determine a BUN/Creatinine ratio, raw activity data may be utilized to determine an average daily activity variable, a HDL/total cholesterol ratio may be determined, and/or a lymphocyte percentage may be determined, among other examples.

Once the training data 108 has been preprocessed, the preprocessed training data may be applied to a cox regression analysis 112 and/or an additive risk model. Using one or more formula defined by the cox regression analysis 112 and/or an additive risk model, the variables may define a proportional hazard of each variable. In other words, the cox regression analysis 112 may determine the amount of time (for example, in years, months, or other period of time) that a particular variable or type of variable adds to users or patients actual age (for example, as a non-limiting example, smoking an amount of cigarettes over a selected period of time adds a number of months to a user's or patient's life). The results of the cox regression analysis 112 and/or the additive risk model may be utilized to produce the trained model 114,

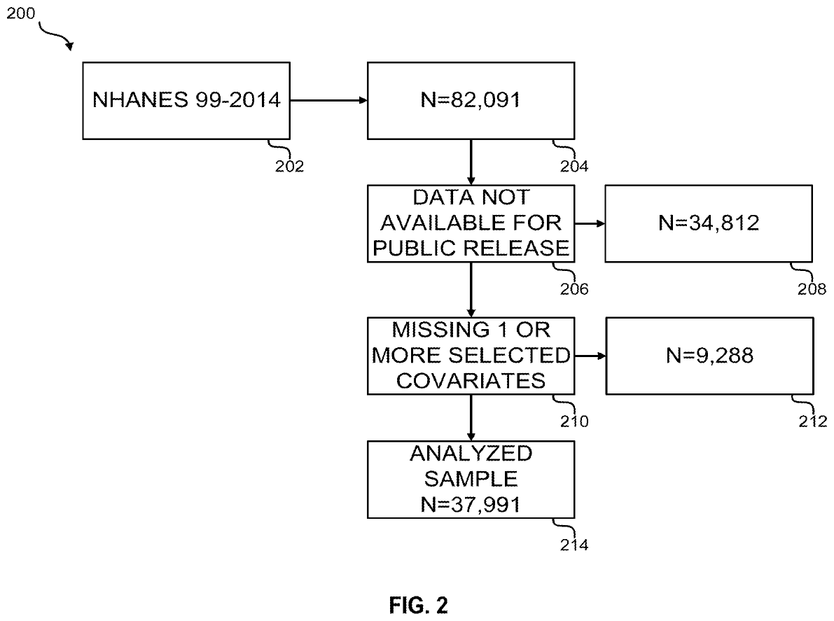

FIG. 2 is a flowchart of a method to select training data to train the machine learning model for determining a biological age or biological age prediction, according to an embodiment of the present disclosure. At block 202, an initial data set may be selected. As illustrated in FIG. 2, the selected data set may be a NHANES data set from the years 1999 to 20214. Other data sets may be selected as initial data sets for training, such as public, private, center for disease control, and/or other government based data sets. Block 204 illustrates the initial amount of data points or amount of subjects in the data set, such as, in a non-limiting example, 82,091 subjects.

Beginning at block 206, the data set may be preprocessed or filtered. As illustrated in block 206, data not available to the public or not available for public release may be removed from the potential training data set. Such an amount, in a non-limiting example, may include about 34,812 subjects. At block 210, subjects missing one or more selected variables or covariates may be removed from the data set. In an non-limiting example, the amount removed may be about 9.288 subjects. Thus, the total subjects or data points in the data set, as illustrated in block 214, to be analyzed may be about 37, 991. As noted, other data sets may be utilized. Further, in an embodiment, larger data sets, or in other embodiments smaller data sets, may be utilized for such an analysis (for example, cox regression analysis or other statistical and/or probabilistic analysis), such as hundreds of thousands, millions, or even more subjects.

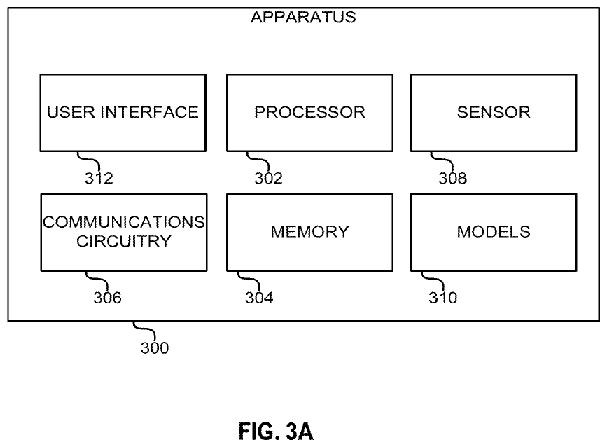

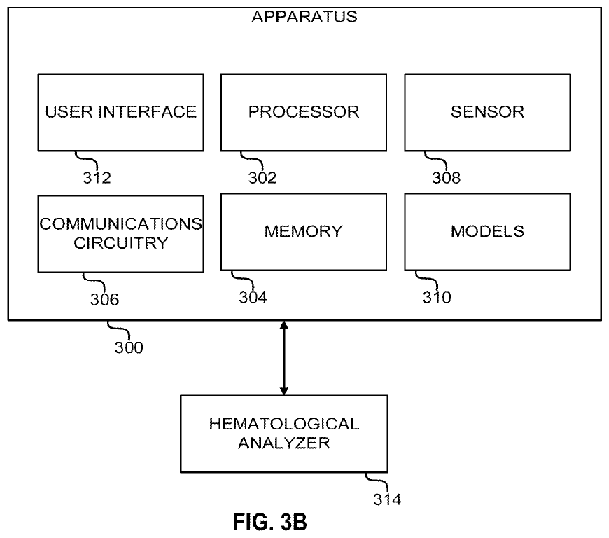

FIG. 3A and FIG. 3B are block diagrams of an apparatus 300 with a model 310 to determine a biological age or biological age prediction, according to an embodiment of the present disclosure. The apparatus 300 will include a processor 302, a memory 304, communications circuitry 306, a sensor 308, a model 310, and/or a user interface 312. The memory 304 may include or store instructions executable by the processor 302.

As used herein, a “processor”, processing resource, or processing circuitry may be a plurality of processors connected together in communication with an electronic communications network. In other embodiments, the processors may be a group of graphical processing units configured to work in parallel as a GPU cluster. A processor may include a single processor device and/or a plurality of processor devices (for example, distributed processors). A processor may be any suitable processor capable of executing/performing instructions. A processor may include a central processing unit (CPU), a semiconductor-based microprocessor, a graphics processing unit (GPU), a field-programmable gate array (FPGA) to retrieve and execute instructions, and/or a real-time processor (RTP) that carries out program instructions to perform the basic arithmetical, logical, and input/output operations required to execute the method of generating a biological age or biological age prediction and/or for providing decision support to healthcare professionals to implement a treatment regimen for a patient based on the biological age or biological age prediction and/or other variables. A processor may include code (for example, processor firmware, a protocol stack, a database management system, an operating system, or a combination thereof) that creates an execution environment for program instructions. Processes and logic flows described herein may be performed by one or more programmable processors executing one or more computer programs to perform functions by operating on input data and generating corresponding output.

In an example, the memory 304 may be a non-transitory machine-readable storage medium. As used herein, a “machine-readable storage medium” may be any electronic, magnetic, optical, or other physical storage apparatus or cyber-physical separation storage to contain or store information such as executable instructions, data, and the like. For example, any machine-readable storage medium described herein may be any of random access memory (RAM), volatile memory, non-volatile memory, flash memory, a storage drive (for example, hard drive), a solid-state drive, any type of storage disc, and the like, or a combination thereof. As noted, the memory 304 may store or include instructions executable by the processor 302.

As used herein, “signal communication” refers to electric communication such as hard wiring two components together or wireless communication, as understood by those skilled in the art. For example, wireless communication may be Wi-Fi®, Bluetooth®, ZigBee, or forms of near field communications. In addition, signal communication may include one or more intermediate controllers or relays disposed between elements in signal communication.

In an embodiment, the model 310 may be configured to produce a biological age or biological age prediction and/or treatment regimen based on the biological age or biological age prediction. The model 310 may be an analytical, statistical, and/or probabilistic model. In another embodiment, the model 310 may be a machine learning model. The machine learning model may be trained such that a trained machine learning model, classifier, predictor, and/or probability is produced. Various machine learning models may be utilized to create the trained machine learning model, classifier, and/or predictor based on the input described herein. Models and methods may include decision trees, random forest models, random forests utilizing bagging or boosting (as in, gradient boosting), neural network methods, support vector machines (SVM), other supervised learning models, other semi-supervised learning models, other unsupervised learning models, or some combination thereof, as will be readily understood by one having ordinary skill in the art. In another embodiment, the model 310 may utilize a large language model, in addition to or rather than other types of machine learning models. In such an embodiment, large data sets may be utilized to train the large language model to interpret user input (for example, demographic variables). The large language model may then be utilized, in addition to or rather than outputs from the other types of machine learning models, to generate the biological age or biological age prediction.

The apparatus 300 will include, in an embodiment, a sensor 308 or plurality of sensors. The sensor 308 or sensors may measure various biometric variables or characteristics. For example, the sensor may measure steps taken, heart rate, blood pressure, temperature, and/or other biometric variables. In such embodiments, the apparatus 300 may be or may comprise a mobile device and/or a wearable device. The apparatus 300 may be worn by a user or patient or held or placed in a pocket of the user or patient. The apparatus 300 may measure, via the sensor, biometric variables as the user or patient wears or holds the apparatus 300. In an embodiment, the apparatus 300 may continuously or substantially continuously measure or determine, via the sensor 308, biometric variables.

The apparatus 300 may determine, prior to gathering biometric variables, a biological age or biological age prediction based on user or patient initialization. The apparatus 300 may prompt user or patient initialization. Such initialization may include prompting a user or patient or automatically gathering demographic variables (for example, from a database connected to the apparatus 300 via the communications circuitry 306 or user input via a user interface 312) and/or hematological variables (for example, from a database connected to the apparatus 300 via the communications circuitry 306, from user input via the user interface 312, and/or from a hematological analyzer 314 as illustrated in FIG. 3B). Once the variables (for example, demographic variables, biometric variables, and/or hematological variables) are received or obtained by the apparatus 300, the apparatus may apply the variables to the model 310. The model 310 may produce a score for each variable. The processor 302 may execute instructions in the memory 304 to generate a biological age or biological age prediction.

Once an initial biological age or biological age prediction has been generated, the apparatus may begin or continue gather or obtain biometric variables via the sensor 308. As additional and/or new biometric variables are obtained and/or as new demographic variables and/or hematological variables are obtained, the apparatus may apply those additional or new variables to the model 310. Thus, a new or updated score and/or biological age or biological age prediction. In another embodiment, the previous biological age or biological age prediction and/or variables may be considered or utilized when generating the updated score and/or biological age or biological age prediction.

Once a biological age or biological age prediction is determined and/or if a user or patient selects a prompt, the apparatus 300 may display the biological age or biological age prediction via the user interface 312. The user interface may include or may be connected to a display (for example, a screen associated with the apparatus 300 and/or a monitor connected to the apparatus 300).

In another embodiment, the apparatus 300 may be configured to determine a treatment regimen based on the generated biological age or biological age prediction. The treatment regimen may include, for example, prescription of a particular medication, a selected diet, selected exercise, and/or other treatments and/or other lifestyle and/or pharmaceutical interventions. The treatment regimen may be displayed via the user interface 312 or transferred to a doctor or medical professional for further review and/or approval.

As used herein, an apparatus, device, or computing device may include one or more of programmable logic controllers (PLCs), programmable automation controllers (PACs), industrial computers, desktop computers, workstations, personal data assistants (PDAs), laptop computers, tablet computers, smart-books, palm-top computers, personal computers, smartphones, wearable devices (for example, headsets, smartwatches, or the like), a server (for example, a rack server, blade server, and/or cluster), and similar electronic devices equipped with at least a processor and any other physical components necessary to perform the various operations described herein.

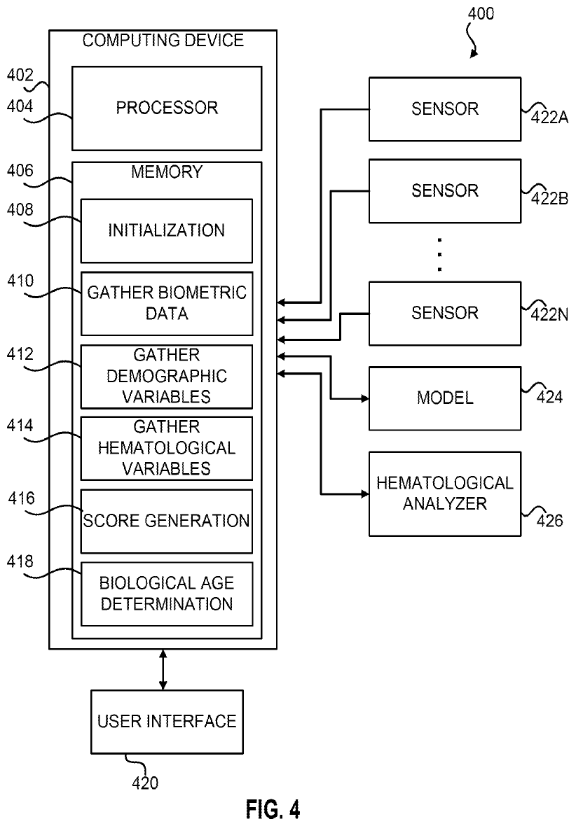

FIG. 4 is a block diagram of a system 400 to determine a biological age or biological age prediction, according to an embodiment of the present disclosure. System 400 may include a computing device 402. The computing device 402 may include a processor 404 and a memory 406. The memory 406 may store instructions executable by the processor 404.

The instructions may include instructions 408 to initialize or to generate an initial biological age or biological age prediction. To generate an initial biological age or biological age prediction, the computing device 402 may execute instructions 410, 412, 414, 416, and/or 418. During initialization, a user or patient may be prompted, via a user interface 420, to enter demographic variables (for example, based on execution of instructions 412). In an embodiment, one or more of the demographic variables may remain the same or similar for an extended period of time or indefinitely. In a non-limiting example, a user or patient's height may, at a particular age, not change. Other demographic variables may change or fluctuate over a user's or patient's life, such as weight. As such, during initialization, many of the demographic variables may be entered and may not be updated for an extended period of time. In another embodiment, execution of instructions 412 may cause the computing device 402 to obtain demographic variables from one or more data sources (for example, a database or other type of storage). For example, the computing device 402 may obtain a user's or patient's electronic health record.

After demographic variables have been gathered, instructions 414 may be executed. In another embodiment, instructions 414 may be executed upon indication that a blood sample has been submitted to a blood analyzer or hematological analyzer 426. The computing device 402 may gather hematological variables from one or more different sources. For example, as noted, the computing device 402 may obtain hematological variables based on analysis of a blood sample by a hematological analyzer 426. In another embodiment, the computing device 402 may obtain hematological variables from other sources (for example, a database or other storage device storing hematological variables).

Once one or more of demographic variables and/or hematological variables are available, the computing device 402 may execute instructions 416 to generate a score. The score may be generated based on application of the one or more of demographic variables and/or hematological variables to the model 424. In an embodiment, each variable may be applied to the model 424. As each variable is applied to the model 424, a score may be produced. Each score may indicate an amount of time (for example, years or portions of a year) to be added to the user's or patients actual age (for example, age acceleration). After each score has been generated, the computing device 402 may aggregate the scores to form an overall score. Once the overall score is available, instructions 418 may be executed to determine the biological age or biological age prediction. The biological age or biological age prediction may be determined based on the overall score and a user's or patient's actual age.

Such instructions may be continuously, substantially continuously, or periodically executed after initialization to generate updated biological ages or biological age predictions. In another embodiment, the biological age or biological age prediction may be updated based on a request provided by the user or patient.

In another embodiment, the biological age or biological age prediction may further be based on application of, in addition to one or more of demographic variables or hematological variables, biometric variables to the model 424. In such embodiments, the computing device 402 may execute instructions 410 to gather the biometric variables. The biometric variables may be gathered or obtained from one or more sensors 422A, 422B, and up to 422N. Each of the one or more sensors 422A, 422B, and up to 422N may sense or measure a biometric variable. The one or more sensors 422A, 422B, and up to 422N may be positioned in or on, included in, and/or may be separate from the computing device 402.

In another embodiment, the computing device 402 may include instructions to update, train, retrain, and/or refine the model. For example, the computing device 402 may be positioned at a hospital or medical facility. As patient mortality status is updated, a set of corresponding variables (for example, the variables used to determine the patient's corresponding biological age or biological age prediction) may be utilized to retrain or refine the model 424. In such examples, the biological age or biological age prediction may be utilized, by the computing device 402, to determine a life expectancy of a patient based on the determined biological age or biological age prediction and actual age of the patient. When a patient dies, the outcome or prediction is known. Thus, variables with a known outcome may be utilized to refine or retrain the model 424.

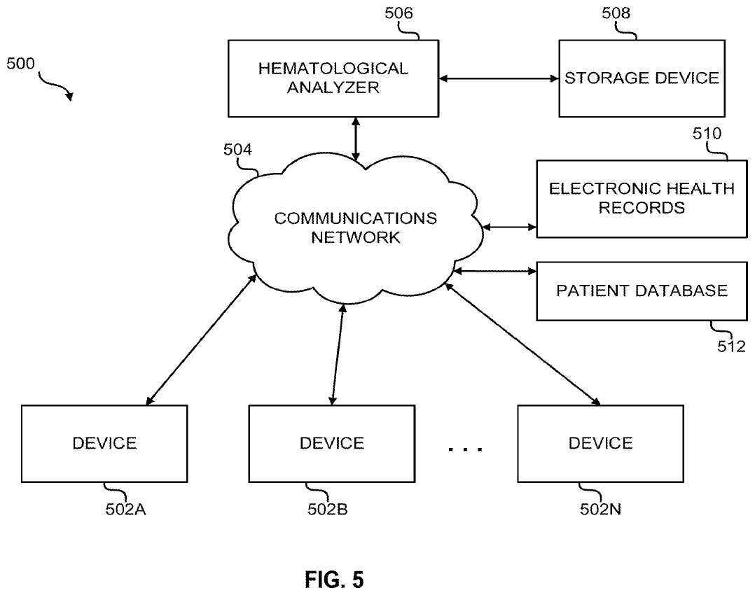

FIG. 5 is another block diagram of a system 500 to determine a biological age or biological age prediction, according to an embodiment of the present disclosure. As illustrated in FIG. 5, the system 500 may include a plurality of devices 502A, 502B, and up to 502N. The plurality of devices 502A, 502B, and up to 502N may comprise different devices, such as a computing device, mobile device, wearable devices, devices including or comprising hematological analyzers, and/or devices positioned at a hospital or medical facility, among other remote and/or distributed devices configured to determine a biological age or biological age prediction of a user or patient.

The plurality of devices 502A, 502B, and up to 502N may each be configured to determine a biological age or biological age prediction of a user or patient, for example, by using a model and one or more of demographic variables, hematological variables, and/or biometric variables. Each of the plurality of devices 502A, 502B, and up to 502N may include a user interface configured to enable a user to input demographic variables. In another embodiment, each of the plurality of devices 502A, 502B, and up to 502N may receive demographic variables from other sources. Each of the plurality of devices 502A, 502B, and up to 502N may connect, via a communications network 504, to other components of the system 500, such as a storage device or database (for example, such as a patient database 512 or a database for storing electronic health records 510).

Each of the plurality of devices 502A, 502B, and up to 502N may include or may connect to, via the communications network 504, a hematological analyzer 506. The hematological analyzer 506 may analyze a blood sample and provide a blood panel and/or hematological variables. The hematological analyzer 506 may be connected to a storage device 508 to store blood panels and/or hematological variables. Thus, each of the plurality of devices 502A, 502B, and up to 502N may obtain a blood panel or hematological variables from the hematological analyzer 506 and/or the storage device 508.

As noted, the plurality of devices 502A, 502B, and up to 502N may also obtain or measure biometric variables, such as via sensors connected to or integrated with each of the plurality of devices 502A, 502B, and up to 502N.

Once one the plurality of devices 502A, 502B, and up to 502N have obtained one or more different variables for a particular user or patient, the one of the plurality of devices 502A, 502B, and up to 502N may determine a biological age or biological age prediction for that particular user or patient. Such a system 500 can be utilized to determine a user's or patient's biological age or biological age prediction or, in other words, the age of the user plus an amount of time based on the variables described herein. Such a biological age or biological age prediction may provide meaningful content for typically difficult to understand or difficult to contextualize data. Further still, the biological age or biological age prediction may be utilized to determine a user's or patient's life expectancy, based on that user's or patient's predicted lifespan or based on an average persons lifespan. Finally, the biological age or biological age prediction may be utilized to determine a treatment regimen for a patient.

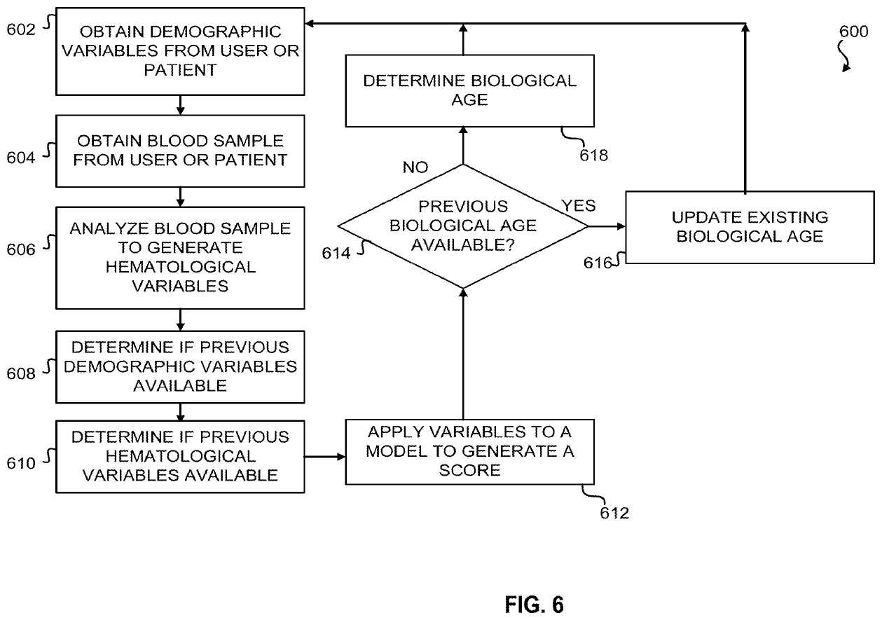

FIG. 6 is a flowchart of a method 600 to utilize the machine learning model for determining a biological age or biological age prediction, according to an embodiment of the present disclosure. The actions of method 600 may be completed within the apparatus 300, system 400, or system 500. Method 600 may be included in one or more programs, protocols, or instructions loaded into the memory of the apparatus or other devices of system 400 or system 500 and executed on one or more corresponding processors. The order in which the operations are described is not intended to be construed as a limitation, and any number of the described blocks may be combined in any order and/or in parallel to implement the methods.

At block 602, a computing device or system may obtain demographic variables from a user or patient. In an embodiment, the user may be a patient, a doctor, or other medical professional. For example, a doctor may enter, via a user interface of a computing device or system, demographic variables for a patient. In an embodiment, the computing device or system may automatically obtain the demographic variables from one or more data sources or databases. In a further embodiment, the user may provide credentials or proof of identity to obtain the demographic variables. Further, the demographic variables may be encrypted. In other words, when a computing device or system obtains the demographic variables, the demographic variables or other data may be encrypted. The computing device or system may utilize one or more cryptographic algorithms, such as a RSA algorithm, a Diffie-Helman algorithm, and/or another cryptographic algorithm, as will be understood by those skilled in the art.

At block 604, a hematological analyzer may receive or obtain a blood sample. A user or patient may provide the blood sample to the hematological analyzer. In an embodiment, the hematological analyzer may be included in or may be integrated with the computing device or system. At block 606, the hematological analyzer may analyze the blood sample to produce or generate a blood panel may analyze the blood samples to generate or determine hematological variables and/or a blood panel.

At block 608, the computing device or system may determine if previous demographic variables are available. The computing device or system may determine whether such data is stored in one or more selected or specified locations (for example, memory). At block 610, the computing device or system may determine if previous hematological variables and/or blood panels are available.

At block 612, the computing device or system may apply the received and/or previously obtained variables to a model to generate a score, such as the demographic variables, previous demographic variables, a blood panel, previous blood panel, hematological variables, and/or previous hematological variables. Other variables and/or data may be applied to a model, such as biometric data. Each variable may be applied to the model. Application of a variable to the model may produce a score. Thus, a plurality of scores may be generated or one score per variable may be generated.

At block 614, the computing device or system may determine whether a previous or previously generated biological age or biological age prediction is available. At block 616, if a previous biological age or biological age prediction is available, the previous biological age or biological age prediction may be updated. The scores generated at block 612 may be utilized to determine the biological age or biological age prediction. At block 618, if a previous biological age or biological age prediction is available, then an initial biological age or biological age prediction may be generated.

In another embodiment, the computing device or system may determine a predicted age or life expectancy of the user or patient. The computing device or system may perform such determinations utilizing the user's or patient's biological age or biological age prediction, actual age, an average life expectancy, and/or other variables,

In another embodiment, the computing device or system may determine a treatment regimen. The computing device or system may determine the treatment regimen based on the biological age or biological age prediction and/or predicted life expectancy, as well as other variables. The treatment regimen may be displayed to the user or patient.

### Example 2

### Materials and Methods

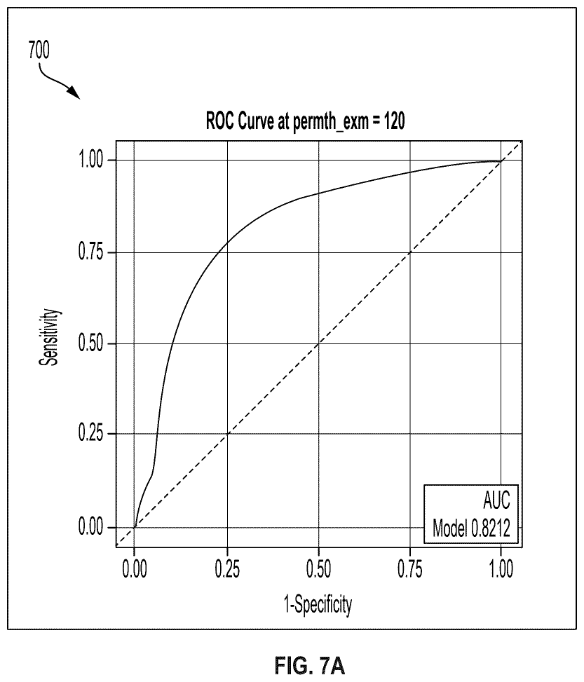

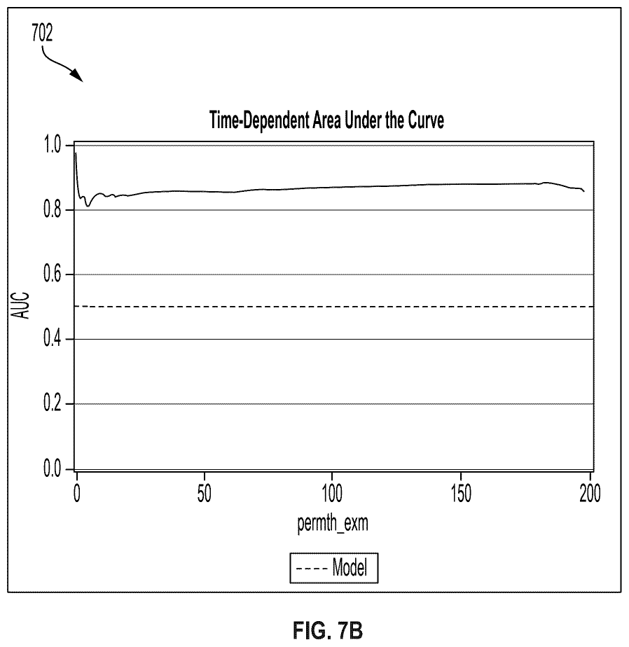

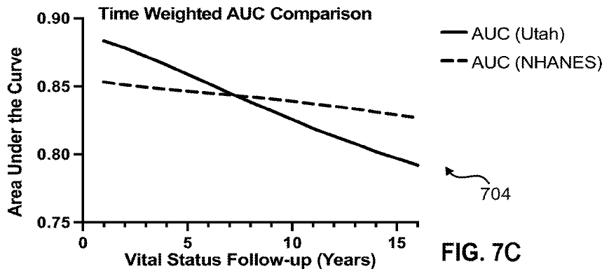

FIG. 7A and FIG. 7B illustrate a receiver operating characteristic (ROC) curve 700 and an area under the ROC (AUC ROC) curve 702 regarding determination of a biological age. To assess the underlying mortality prediction capability of the composite model from which the biological age is derived, a 10-fold cross validation procedure was performed. 10% of the total analyzed sample was selected from a dataset (for example, such as the final NHANES data set as illustrated in FIG. 2) at random, serving as a test dataset from which a AUC ROC 702 value could be generated. This process was repeated ten times, with a random 10% of the sample selected each time. 10 AUC values were calculated from 10 randomly generated test datasets. From these, a mean AUC value can be reported for the overall composite model. This process was repeated using multiple permutations of variables to determine the durability of the model's predictive power in hypothetical clinical scenarios where the values of all variables or covariates may not be known.

2,552 deaths were observed over the course of the mortality follow up period. Underlying cause of death distribution within the analyzed sample is displayed in Table 1. Mortality event time distribution is displayed in Table 2. The data for such a multi-decade survival analysis may skew heavily to the right.

TABLE 1
Underlying Cause of Death Frequencies

| | |
|---|---|
| Cause of Death Category | Frequency (%) |

| | | |
|---|---|---|
| All other causes | 1,947 | (41.9) |
| Malignant neoplasms | 1,006 | (21.6) |
| Diseases of heart | 799 | (17.2) |
| Chronic lower respiratory | 180 | (3.9) |
| Cerebrovascular diseases | 179 | (3.9) |
| Accidents (unintentional injuries) | 163 | (3.5) |
| Diabetes mellitus | 122 | (2.6) |
| Alzheimer's disease | 110 | (2.4) |
| Nephritis, nephrotic syndrome and nephrosis | 74 | (1.6) |
| Influenza and pneumonia | 71 | (1.5) |

TABLE 2
Mortality Event Time Distribution

| Quantile | Frequency (%) | |
|---|---|---|
| 100% Max | 937 | (36.7) |
| 99% | 901 | (35.3) |
| 95% | 194 | (7.6) |
| 90% | 181 | (7.1) |
| 75% Q3 | 142 | (5.6) |
| 50% Median | 92 | (3.6) |
| 25% Q1 | 50 | (2.0) |
| 10% | 26 | (1.0) |
| 5% | 18 | (0.7) |
| 1% | 12 | (0.5) |

41 of the 46 analyzed hematological and demographic variables displayed a significant relationship with mortality risk in a univariate analysis. 20 variables remained independently and significantly associated with mortality risk in a multivariable analysis. These variables, along with summary statistics, are displayed in Table 3 and are grouped by variable/laboratory procedure category. Variables are presented with the appropriate descriptive statistic reference value needed for calculating the biological age described herein.

Table 4 displays variable names, hazard ratios, confidence intervals, p-values, and age ratios for the 20 variables. They all displayed a linear relationship with mortality risk, except for chloride and mean platelet volume. These variables are therefore reported in quintiles.

Table 5 displays the model's covariates ranked by standardized hazard ratio. Although not a common method of ranking of covariates, it helps account for differences in units and breadth of clinical range between variables. This metric is not used for CompositeAge calculations, but rather a visualization of each variable's potential contribution to the composite risk model.

TABLE 3
Hematological and Demographic Variables with
Summary Statistics: Reference Values for LifespanACCEL
and CompositeAge Calculations.

| | Summary |
|---|---|
| Variable Name | Statistic |
| Smoked at least 100 cigarettes in life (Y/N) | |
| Yes | 17596 (46.32) |
| No | 20395 (53.68) |
| Sex | |
| Male | 18326 (48.24) |
| Female | 19665 (51.76) |
| Body Mass Index (kg/m²) | 28.736 ± 6.632 |
| Age at Screening | 48.986 ± 18.326 |
| Glycohemoglobin: (%) | 5.666 ± 1.042 |
| Triglycerides (mg/dL) | 149.261 ± 112.515 |
| Cholesterol, total (mg/dL) | 196.562 ± 41.833 |
| Uric acid (mg/dL) | 5.408 ± 1.457 |
| Red cell distribution width (%) | 12.997 ± 1.273 |
| White blood cell count (SI) | 7.261 ± 2.423 |
| Mean cell volume (fL) | 89.634 ± 5.691 |
| Mean platelet volume Quintile 1: <7.5 (fL) | 7871 (20.72) |
| Mean platelet volume Quintile 2: 7.5-7.9 (fL) | 8158 (21.47) |
| Mean platelet volume Quintile 3: 9-8.3 (fL) | 6962 (18.33) |
| Mean platelet volume Quintile 4: 8.4-8.9 (fL) | 8136 (21.42) |
| Mean platelet volume Quintile 5: >8.9 (fL) | 6864 (18.07) |
| Lactate Dehydrogenase (LDH) (U/L) | 132.697 ± 32.812 |
| Alkaline phosphotase (U/L) | 71.199 ± 26.723 |
| Potassium (mmol/L) | 4.001 ± 0.349 |
| Gamma-glutamyl Transferase (GGT) (U/L) | 29.575 ± 43.450 |
| Blood Urea Nitrogen Creatinine Ratio | 15.562 ± 5.722 |
| Alanine Transaminase (ALT) (U/L) | 25.493 ± 25.725 |
| Aspartate Aminotransferase (AST) (U/L) | 25.639 ± 19.428 |
| Chloride Quintile 1: <101.1 (mmol/L) | 7709 (20.29) |
| Chloride Quintile 2: 101.1-103 (mmol/L) | 9443 (24.86) |
| Chloride Quintile 3: 103.1-104 (mmol/L) | 5856 (15.41) |
| Chloride Quintile 4: 104.1-105.9 (mmol/L) | 5581 (14.69) |
| Chloride Quintile 5: >106 (mmol/L) | 9402 (24.75) |

TABLE 4
Cox Proportional Hazards Regression Multivariable Analysis: (Hazard
Ratios, 95% Confidence Intervals, p-values, and Age Ratios)

| | | | | p- | Age |
|---|---|---|---|---|---|
| Variable Name | HR | LL | UL | value | Ratio |
| Age at Screening | 1.07 | 1.067 | 1.072 | <.0001 | |
| Alkaline phosphotase (U/L) | 1.003 | 1.002 | 1.003 | <.0001 | 0.044 |
| ALT (U/L) | 0.996 | 0.994 | 0.999 | 0.0009 | −0.059 |
| AST (U/L) | 1.004 | 1.001 | 1.006 | 0.0036 | 0.059 |
| Blood Urea Nitrogen ratio | 0.986 | 0.978 | 0.989 | 0.0002 | −0.208 |
| Body Mass Index (kg/m²) | 0.984 | 0.978 | 0.993 | <.0001 | −0.238 |
| Cholesterol, total (mg/dL) | 0.999 | 0.998 | 1 | 0.0037 | −0.015 |
| Gender | 1.31 | 1.186 | 1.383 | <.0001 | 3.991 |
| GGT (U/L) | 1.001 | 1 | 1.001 | <.0001 | 0.015 |
| Glycohemoglobin: (%) | 1.061 | 1.021 | 1.104 | 0.0031 | 0.875 |
| LDH (U/L) | 1.002 | 1.002 | 1.003 | <.0001 | 0.030 |
| Mean cell volume (fL) | 1.074 | 1.017 | 1.163 | 0.0224 | 1.055 |
| Potassium (mmol/L) | 1.224 | 1.142 | 1.335 | <.0001 | 2.987 |
| Red cell distribution width (%) | 1.124 | 1.104 | 1.146 | <.0001 | 1.728 |
| Smoked at least 100 cigarettes in | 1.301 | 1.224 | 1.387 | <.0001 | 3.889 |
| life | | | | | |
| Triglycerides (mg/dL) | 1.001 | 1 | 1.001 | 0.0008 | 0.015 |
| Uric acid (mg/dL) | 1.037 | 1.015 | 1.062 | 0.0015 | 0.537 |
| Mean Platelet Volume Quintile 0 | 1.348 | 0.998 | 1.208 | <.0001 | 4.414 |
| vs 2 | | | | | |
| Mean Platelet Volume Quintile 1 | 1.042 | 1.012 | 1.221 | 0.4257 | 0.608 |
| vs 2 | | | | | |
| Mean Platelet Volume Quintile 3 | 0.95 | 0.95 | 1.149 | 0.3976 | −0.758 |
| vs 2 | | | | | |
| Mean Platelet Volume Quintile 4 | 0.921 | 0.887 | 1.086 | 0.148 | −1.216 |
| vs 2 | | | | | |
| Chloride Quintile 1 vs 3 | 1.063 | 1.227 | 1.52 | 0.2056 | 0.903 |
| Chloride Quintile 2 vs 3 | 1.097 | 0.945 | 1.158 | 0.0547 | 1.368 |
| Chloride Quintile 4 vs 3 | 1.058 | 0.84 | 1.064 | 0.247 | 0.833 |
| Chloride Quintile 5 vs 3 | 1.025 | 0.817 | 1.021 | 0.6277 | 0.365 |
| White blood cell count 1000 | 1.024 | 1.018 | 1.034 | <.0001 | 0.351 |
| cells/ul | | | | | |

TABLE 5
Standardized Hazard Ratios

| Variable Name | Standardized Hazard Ratio |
|---|---|
| Triglycerides (mg/dL) | 112.627 |
| GGT (U/L) | 43.494 |
| Cholesterol, total (mg/dL) | 41.791 |
| LDH (U/L) | 32.877 |
| Alkaline phosphotase (U/L) | 26.803 |
| ALT (U/L) | 25.622 |
| Age at Screening | 19.609 |
| AST (U/L) | 19.506 |
| Body Mass Index (kg/m²) | 6.526 |
| Mean cell volume (fL) | 6.112 |
| Blood Urea Nitrogen ratio | 5.642 |
| White blood cell count 1000 cells/ul | 2.481 |
| Uric acid (mg/dL) | 1.511 |
| Red cell distribution width (%) | 1.430 |
| Mean Platelet Volume Quintile 0 vs 2 | 1.348 |
| Gender | 1.310 |
| Smoked at least 100 cigarettes in life | 1.301 |
| Glycohemoglobin: (%) | 1.105 |
| Chloride Quintile 1 vs 2 | 1.097 |
| Chloride Quintile 0 vs 2 | 1.063 |
| Chloride Quintile 3 vs 2 | 1.058 |
| Mean Platelet Volume Quintile 1 vs 2 | 1.042 |
| Chloride Quintile 4 vs 2 | 1.025 |
| Mean Platelet Volume Quintile 3 vs 2 | 0.950 |
| Mean Platelet Volume Quintile 4 vs 2 | 0.921 |
| Potassium (mmol/L) | 0.427 |

All-cause mortality predictive power was assessed using Harrell's Concordance Statistic and the results are shown in Table 6. Since this is intended as a clinical model and variables may often be missing in clinical settings, multiple permutations of the model were tested. The AUC values given are time weighted averages. The first permutation is age alone, which yields a 0.8223 AUC. The remaining permutations have AUC values as follows: Age plus demographic variables=0.8281, Age, demographic variables, and differential CBC markers=0.8413, Age, demographics, CBC markers, CMP markers, and uric acid=0.8515, and the full model containing all 20 covariates=0.8516. Lastly, a permutation composed of all covariates except for age results in an AUC of 0.8032.

FIG. 7A displays the cross-validation ROC curve of the full composite model at 10-year mortality follow up. The AUC over time is illustrated in more detail in FIG. 7B.

TABLE 6
Concordance Statistics for Multiple Permutations
of the Composite Mortality Model

| Model Permutation | AUC | Concordance | Discordance |
|---|---|---|---|
| Age | 0.8223 | 87970762 | 18400465 |
| Age + Sex + BMI + Smoking | 0.8281 | 89379834 | 18552531 |
| +5-part Differential CBC Markers | 0.8413 | 90799571 | 17133118 |
| +CMP markers + Uric Acid | 0.8515 | 91908693 | 16023996 |
| +Hemoglobin A1c | 0.8516 | 91918898 | 16013791 |
| Full Model Without Age | 0.8032 | 86696358 | 21236331 |

FIG. 7C illustrates an AUC ROC curve 704 regarding vital status follow-up. The AUC ROC curve 704 illustrates the comparison of the trained model's or the additive risk model's predictive performance over 16 years of follow up in the training dataset (such as NHANES, as illustrated) compared to an external testing dataset (Utah Centre d'Etudes du Polymorphisme Humain). FIG. 7C demonstrates the ability to train a model in a training dataset of demographic, biometric, and hematological variables, then use that model to make accurate vital status predictions in an external sample. In the example above, the model maintained excellent external validity (AUC>0.80) for more than 14 years following data acquisition. The relative mortality risk predictions derived from the model can then be regressed onto age to generate accurate biological age predictions which are patient and clinician relevant.

Although specific terms are employed herein, the terms are used in a descriptive sense only and not for purposes of limitation. Embodiments of systems and methods have been described in considerable detail with specific reference to the illustrated embodiments. However, it will be apparent that various modifications and changes can be made within the spirit and scope of the embodiments of systems and methods as described in the foregoing specification, and such modifications and changes are to be considered equivalents and part of this disclosure.

What is claimed is:

1. A method for generating a biological age, the method comprising:
receiving one or more of hematological variables or demographic variables corresponding to a user;
applying each one the one or more of the hematological variables or the demographic variables to a model to thereby generate a score; and
determining a biological age for the user based on the score.

2. The method of claim 1, wherein the model comprises one or more of a composite model, a cox proportional hazards regression model, an unsupervised model, a supervised model, or a machine learning model.

3. The method of claim 1, further comprising, prior to application of each of the one or more of the hematological variables or the demographic variables to the model, pre-processing each of the one or more of the hematological variables or the demographic variables.

4. The method of claim 1, wherein pre-processing includes formatting the one or more of the hematological variables or the demographic variables to a format applicable to the model and filtering the one or more of the hematological variables or the demographic variables to remove non-applicable or outlying variables or anomalies.

5. The method of claim 1, wherein generation of the score includes generation of an age ratio metric for each variable, each age ratio metric defined by a beta coefficient of each variable divided by a beta coefficient of age.

6. The method of claim 5, wherein determination of the biological age is based on each age ratio metric and a current age of the user.

7. The method of claim 5, wherein each age ratio metric is based on variables comprising one or more of whether the user smoked a selected number of cigarettes in the user's lifetime, a gender of the user, a body mass index of the user, a current age of the user, a glycohemoglobin percentage of a user's blood sample, a triglyceride content of the user's blood sample, a total cholesterol of the user's blood sample, a uric acid content of the user's blood sample, a red cell distribution width percentage of the user's blood sample, a white blood cell count of the user's blood sample, a mean cell volume of the user's blood sample, a mean platelet volume quintile 1 of the user's blood sample, a mean platelet volume quintile 2 of the user's blood sample, a mean platelet volume quintile 3 of the user's blood sample, a mean platelet volume quintile 4 of the user's blood sample, a mean platelet volume quintile 5 of the user's blood sample, a lactate dehydrogenase content of the user's blood sample, an alkaline phosphatase content of the user's blood sample, a potassium content of the user's blood sample, a gamma-glutamyl transferase of the user's blood sample, a blood urea nitrogen creatine ratio of the user's blood sample, an alanine transaminase content of the user's blood sample, an aspartate aminotransferase content of the user's blood sample, a chloride quintile 1 of the user's blood sample, a chloride quintile 2 of the user's blood sample, a chloride quintile 3 of the user's blood sample, a chloride quintile 4 of the user's blood sample, or a chloride quintile 5 of the user's blood sample.

8. The method of claim 1, wherein the model is a trained model, and wherein training data for training the model includes one or more publicly available datasets, each of the one or more publicly available datasets including hematological variables and demographic variables.

9. The method of claim 8, wherein each of the one or more publicly available datasets include cause of death data for one or more subjects.

10. The method of claim 9, further comprising validating the trained model via a cross validation procedure with data from the one or more publicly available datasets which include hematological variables, demographic variables, and cause of death data.

11. The method of claim 1, wherein the biological age is further based on one or more of previously received hematological data, previously received demographic data, or a previously determined biological age.

12. The method of claim 1, further comprising determining a treatment regimen based on the biological age and a user's current age.

13. A method for generating a biological age, the method comprising:
receiving one or more of one or more hematological variables or one or more demographic variables corresponding to a user;
determining availability of one or more of (1) previously received hematological variables, (2) previously received demographic variables corresponding to the user, or (3) previously generated biological ages;
applying each one of the one or more of (1) one or more hematological variables, (2) one or more demographic variables, (3) the previously received hematological variables, or (4) the previously received demographic variables corresponding to the user to thereby generate a new biological age of the user; and
determining an updated biological age based on the new biological age and the previously generated biological ages.

14. The method of claim 13, wherein a hematological analyzer generates the one or more hematological variables based on a blood sample of the user.

15. The method of claim 13, wherein the user provides the one or more demographic variables via a user interface.

16. The method of claim 15, further comprising, in response to determination of the updated biological age:
determining a treatment regimen based on the updated biological age, the new biological age, and a previously generated biological age, and
displaying one or more of the updated biological age, the new biological age, or the previously generated biological age.

17. The method of claim 16, further comprising displaying the one or more hematological variables or one or more demographic variables, and wherein the treatment regimen is further based on the one or more hematological variables or one or more demographic variables.

18. The method of claim 17, further comprising displaying a difference between the biological age and a current age of the user.

19. An apparatus for generating a biological age, the apparatus comprising:
at least one biometric sensor configured to substantially continuously monitor and gather biometric data of a user;
a user interface;
a processor; and
a non-transitory machine-readable storage medium storing processor-executable instructions that, when executed by the processor, cause the processor to:
in response to a determination that a user profile includes an existing biological age:
apply gathered biometric data to a model to thereby generate a new biological age, determine an updated biological age based on the new biological age and an existing biological age, and
display the updated biological age to the user interface.

20. The apparatus of claim 19, wherein the apparatus comprises a mobile device.

21. The apparatus of claim 20, wherein the mobile device comprises a wearable device.

22. The apparatus of claim 21, wherein the non-transitory machine-readable storage medium includes executable instructions, when executed by the processor, to cause the processor to:
request demographic variables of the user;
in response to reception of the demographic variables, apply received demographic variables, in addition to the gathered biometric data, to the model to thereby generate the new biological age; and
determine the updated biological age based on the new biological age and the existing biological age.

23. The apparatus of claim 22, wherein the non-transitory machine-readable storage medium includes executable instructions, when executed by the processor, to cause the processor to, prior to determination of the updated biological age and if the existing biological age is not available:
prompt the user to submit a current actual age; and
in response to reception of the current actual age, determine the updated biological age based on the new biological age and the current actual age.

24. The apparatus of claim 19, wherein the non-transitory machine-readable storage medium includes executable instructions, when executed by the processor, to cause the processor to, prior to determination of the updated biological age:
prompt the user to initialize the apparatus, wherein initialization includes one or more of entry of demographic variables or establishment of a connection with a data source including hematological variables.

25. The apparatus of claim 24, wherein the non-transitory machine-readable storage medium includes executable instructions, when executed by the processor, to cause the processor to, prior to determination of the updated biological age:
apply one or more of the demographic variables or hematological variables, in addition to gathered biometric data, to the model to determine the new biological age.

26. An apparatus for generating a biological age, the apparatus comprising:
a user interface configured to receive demographic variables corresponding to each of a plurality of patients;
a communications circuitry configured to connect with an analyzer, the analyzer configured to receive hematological variables corresponding to each of the plurality of patients;
a processor; and
a non-transitory machine-readable storage medium storing processor-executable instructions that, when executed by the processor, cause the at least one processor to:
apply received demographic variables and received hematological variables for one of the plurality of patients to a model to thereby generate a score, and
determine a biological age based on the score.

27. The apparatus of claim 26, wherein the apparatus is positioned proximate a medical practitioners office or a hospital.

28. The apparatus of claim 26, wherein the user interface is configured to display the biological age.

29. The apparatus of claim 28, wherein the non-transitory machine-readable storage medium includes executable instructions, when executed by the processor, to cause the processor to determine a treatment regimen based on the biological age, and wherein the user interface is configured to display the treatment regimen.

30. An apparatus for generating a biological age, the apparatus comprising:
a user interface configured to receive demographic variables corresponding to each of a plurality of patients;
a hematological analyzer configured to receive a biological sample and generate hematological variables;
a processor; and
a non-transitory machine-readable storage medium storing processor-executable instructions that, when executed by the processor, cause the at least one processor to:
apply received demographic variables and hematological variables for one of the plurality of patients to a model to thereby generate a score, and
determine a biological age based on the score.

31. The apparatus of claim 30, wherein the user interface is configured to receive biometric measurements.

32. The apparatus of claim 31, wherein the biometric measurements include one or more of weight, height, or blood pressure.

33. The apparatus of claim 32, wherein generation of the score is further based on application of the biometric measurements to the model.

34. The apparatus of claim 30, wherein the user interface receives the demographic variables from electronic health records.

35. The apparatus of claim 34, wherein a database in signal communication with the user interface stores the electronic health records.

36. A system for generating a biological age, the system comprising:
a hematological analyzer configured to receive a biological sample and generate hematological variables;
a device in signal communication with the hematological analyzer and including:
a user interface configured to receive demographic variables corresponding to each of a plurality of patients;
a processor; and
a non-transitory machine-readable storage medium storing processor-executable instructions that, when executed by the processor, cause the at least one processor to:
apply hematological variables from the hematological analyzer and received demographic variables for one of the plurality of patients to a model to thereby generate a score, and
determine a biological age based on the score.

37. The system of claim 36, wherein a score is generated for each hematological variable and each demographic variable,
wherein the non-transitory machine-readable storage medium includes executable instructions, when executed by the processor, to cause the processor to aggregate each score to form an aggregate score, and
wherein the biological age is determined based on the aggregate score.
